(19) United States
(12) Patent Application Publication
(10) Pub. No.: US 2014/0010882 A1
(43) Pub. Date: Jan. 9, 2014

(54) PHARMACEUTICAL COMPOSITION FOR TREATING A DISEASE IN THE ORAL CAVITY COMPRISING REBAMIPIDE

(71) Applicants: Takakuni Matsuda, Osaka (JP); Nobutomo Sako, Osaka (JP); Takako Nakashima, Osaka (JP); Kazushi Sakurai, Osaka (JP)

(72) Inventors: Takakuni Matsuda, Osaka (JP); Nobutomo Sako, Osaka (JP); Takako Nakashima, Osaka (JP); Kazushi Sakurai, Osaka (JP)

(21) Appl. No.: 14/006,777

(22) PCT Filed: Mar. 22, 2012
(86) PCT No.: PCT/JP2012/058280

§ 371 Date: Sep. 23, 2013

(30) Foreign Application Priority Data
Mar. 24, 2011 (JP) 2011-066353

(51) Int. Cl. A61K 9/10; A61K 31/4704

(52) U.S. Cl. CPC A61K 9/10; A61K 31/4704
USPC 424/489; 514/312

(57) ABSTRACT

The present invention is directed to a pharmaceutical composition comprising rebamipide having a mean particle size of less than 500 nm, a dispersing agent, and a viscosity enhancing agent wherein the viscosity enhancing agent has no aggregative action for the rebamipide particles, which is used as a gargle or a liquid preparation for swish and swallow comprising rebamipide for preventing and/or treating stomatitis caused by radiotherapy.

### TECHNICAL FIELD

The present invention relates to a pharmaceutical composition suitable for treating disease(s) in the oral cavity or in the pharynx, in particular, mucosal disorder in the oral cavity, which comprises rebamipide [chemical name: 2-(4-chlorobenzoylamino)-3-(2-oxo-1,2-dihydro-quinolin-4-yl)propanoic acid] as an active ingredient whose mean particle size is less than 500 nm (preferably, the mean particle size of less than 300 nm); a method for preparing the composition; and use thereof.

### BACKGROUND ART

It is known that rebamipide or a salt thereof which is an active ingredient in the present pharmaceutical composition is useful as a medicament for treating gastric inflammation/gastric ulcer. In addition, it is also disclosed that rebamipide is useful for treating dry eye, i.e., xerophthalmia (Patent Reference 1), and a pharmaceutical composition as a saliva secretion stimulant comprising rebamipide is also known (Patent Reference 2). Furthermore, Patent Reference 3 discloses an oral formulation comprising rebamipide having an action for inhibiting the production of interleukin-8, wherein the treatment coverage includes the treatment of stomatitis.

In the meantime, main methods for treating head and neck cancer include surgical resection and radiation, or a combination therapy thereof with an anticancer agent. In case of radiation therapy, mucosal disorder in the oral cavity (stomatitis) can frequently happen as a side-effect. When the symptom is severe, the patient can hardly eat food, at last the radiation therapy can be forced to stop. Thus, stomatitis has been a problem in the therapy of head and neck cancer, but there has been no useful method for treating such side-effect.

It was already reported that gargling with a gargle comprising rebamipide before radiotherapy is effective for preventing stomatitis caused by radiotherapy (non-Patent Reference 1). However, the case report was only a suggestion for preventive effect of rebamipide for stomatitis, in which the concentration of the rebamipide in the gargle was relatively low, and there are still problems to be solved about the dosing volume and the number of medication.

There are some other disclosures that rebamipide is used as a liquid formulation, for example, a liquid formulation wherein a rebamipide tablet is crashed and then suspended in water or carboxymethylcellulose sodium salt solution, and a liquid formulation wherein a rebamipide tablet is suspended in a mixed solution of ALKOX® (polyethylene oxide) and Inagel® (non-Patent Reference 2). In the above liquid formulations, however, the concentration of rebamipide was low, i.e., 300 mg/300 mL or 600 mg/300 mL (i.e., 1 mg/mL or 2 mg/mL), and about 50 mL per one gargling, and furthermore the rebamipide used therein was larger particles, which are contained in the commercial tablet. In addition, ALKOX® (polyethylene oxide) has a problem to use as a pharmaceutical additive because it is an industrial additive.

Patent Reference 4 discloses a formulation example as a spray formulation for treating stomatitis (rebamipide 0.2 mg/mL) which was prepared by mixing 100 mg of rebamipide, 2 g of Inagel® (F-13), and 5 g of ALKOX® (E-30) in sterile purified water, adding parabens and ethanol thereto, and adjusting the volume to 500 mL, but in which the concentration of rebamipide was low, and additionally the particle size of rebamipide was not studied in the reference.

In addition, Patent Reference 5 discloses a suspension of rebamipide microparticles prepared by mixing at least one of the compounds selected from water-soluble polymers and surfactants, an aqueous acidic solution, and an aqueous solution containing a water-soluble salt of rebamipide, whose transparency was improved, but the patent reference discloses only the composition for ophthalmic use which does not include a viscosity enhancing agent.

Patent Reference 6 discloses a hydrogel suspension which comprises a suspension of a fine particle prepared by mixing at least, one of the compounds selected from water-soluble polymers and surfactants, an acidic aqueous solution, and an aqueous solution containing a water-soluble salt of rebamipide; and high molecular weight hydroxypropylmethyl cellulose or methylcellulose.
[Patent Reference 1] JP 9-301866 A (1997)
[Patent Reference 2] JP 2006-528662 T
[Patent Reference 3] JP 8-012578 A (1996)
[Patent Reference 4] JP 2002-255852 A
[Patent Reference 5] WO 2006/052018
[Patent Reference 6] WO 2007/132907
[non-Patent Reference 1] Keiji KAWATA, et al., Journal of New Remedies & Clinics, 50:273-280, 2001
[non-Patent Reference 2] Takehisa HANAWA, et al., The Pharmaceuticals Monthly, 50:1717-1724

### SUMMARY OF INVENTION

### Problem to be Solved by the Invention

In order to generally use a gargle or a liquid preparation for swish and swallow comprising rebamipide for treating stomatitis caused by radiotherapy or cancer chemotherapy, it is necessary to increase the concentration of rebamipide and the adherability of rebamipide to oral mucosa. However, a liquid formulation having a high concentration of rebamipide is required to secure the dispersibility and prevent the aggregation. Thus, it has been desired to develop a useful gargle or a liquid preparation for swish and swallow comprising rebamipide having a high effect for treating stomatitis, which is easy for a patient to use.

### Means to Solve the Problem

The present inventors have extensively studied in order to solve the above-mentioned problems, and have found that a pharmaceutical composition as an aqueous suspension comprising 10 mg/mL-50 mg/mL rebamipide having a mean particle size of less than 500 nm as an active ingredient, at least one dispersing agent, and at least one viscosity enhancing agent, wherein the viscosity enhancing agent has no aggregative action for the rebamipide particles having a mean particle size of less than 500 nm, and the viscosity of the liquid preparation is in a range of 10 mPa·s-500 mPa·s has a potent therapeutic benefit for stomatitis. Based upon the new findings, the present invention has been completed.

The present invention includes the following embodiments.

[1] A pharmaceutical composition comprising 10 mg/mL-50 mg/mL rebamipide having a mean particle size of less than 500 nm as an active ingredient, at least one dispersing agent, and at least one viscosity enhancing agent, wherein the viscosity of the liquid preparation is in a range of 10 mPa·s-500 mPa·s.

[2] The pharmaceutical composition of [1] wherein the mean particle size of rebamipide is less than 300 nm, the content of rebamipide is 20 mg/mL-40 mg/mL, and the viscosity of the liquid preparation is in a range of 20 mPa·s-300 mPa·s.

[3] The pharmaceutical composition of [1] or [2] wherein the dispersing agent comprises at least one ingredient selected from the group consisting of polyvinylpyrrolidone, hydroxypropylmethylcellulose, polyoxyethylene polyoxypropylene glycol, and carboxymethylcellulose sodium.

[4] The pharmaceutical composition of [3] wherein the dispersing agent comprises polyvinylpyrrolidone.

[5] The pharmaceutical composition of [4] wherein the dispersing agent comprises polyvinylpyrrolidone K25 and/or polyvinylpyrrolidone K30.

[6] The pharmaceutical composition of any one of [1]-[5] wherein the viscosity enhancing agent comprises polyvinylpyrrolidone K90.

[7] The pharmaceutical composition of any one of [1]-[5] wherein the viscosity enhancing agent comprises pullulan.

[8] The pharmaceutical composition of any one of [1]-[5] wherein the viscosity enhancing agent comprises polyvinylpyrrolidone K90 and pullulan.

[9] The pharmaceutical composition of [8] wherein the viscosity enhancing agent comprises 5 mg/mL-30 mg/mL polyvinylpyrrolidone K90 and 10 mg/mL-30 mg/mL pullulan.

[10] The pharmaceutical composition of any one of [1]-[9] wherein the viscosity enhancing agent has no aggregative action for the rebamipide particles.

[11] The pharmaceutical composition of any one of [1]-[9] which is prepared via the following steps

mixing at least one dispersing agent, an aqueous acid solution, an aqueous solution comprising a water-soluble rebamipide salt, and optional other ingredient(s) or solvent(s) to prepare an aqueous suspension comprising rebamipide having a mean particle size of less than 500 nm, and then

adding a viscosity enhancing agent thereto.

[12] The pharmaceutical composition of [11] which is prepared via the following steps

mixing at least one dispersing agent, an aqueous acid solution, an aqueous solution comprising a water-soluble rebamipide salt, and optional other ingredient(s) or solvent(s) to prepare an aqueous suspension comprising rebamipide having a mean particle size of less than 500 nm,

adding a base thereto to adjust pH of the aqueous suspension to 3-7,

dispersing and/or dialyzing the aqueous suspension,

adjusting pH of the aqueous suspension to 5-7, and then

adding a viscosity enhancing agent thereto.

[13] The pharmaceutical composition of any one of [1]-[12] wherein the mean particle size of rebamipide is less than 200 nm.

[14] The pharmaceutical composition of any one of [1]-[13] wherein the shape of rebamipide is a uniform needle crystal having the longest diameter of less than 1000 nm and the shortest diameter of less than 60 nm, provided that the longest diameter/the shortest diameter is more than 3.

[15] The pharmaceutical composition of any one of [1]-[14], further comprising a parahydroxybenzoate derivative as a preserving agent (antiseptic agent).

[16] The pharmaceutical composition of any one of [1]-[15], further comprising an isotonic agent, a sweetening agent, and/or a flavor.

[17] The pharmaceutical composition of [16] comprising stevia as a sweetening agent.

[18] The pharmaceutical composition of any one of [1]-[17] which is in the form of aqueous suspension.

[19] A method for preparing the pharmaceutical composition of any one of [1]-[18], comprising

mixing at least one dispersing agent, an aqueous acid solution, an aqueous solution comprising a water-soluble rebamipide salt, and optional other ingredient(s) or solvent(s) to prepare an aqueous suspension comprising rebamipide having a mean particle size of less than 500 nm,

adding a base thereto to adjust pH of the aqueous suspension to 3-7,

dispersing and/or dialyzing the aqueous suspension,

adjusting pH of the aqueous suspension to 5-7, and then

adding a viscosity enhancing agent thereto and optionally adding a preserving agent (antiseptic agent), an isotonic agent, a sweetening agent, and/or a flavor thereto.

[20] A method for preventing and/or treating mucosal disorder in the oral cavity, comprising administering the pharmaceutical composition of any one of [1]-[18] in the oral cavity.

[21] A method for preventing and/or treating mucosal disorder in the oral cavity and/or mucosal disorder in the pharynx, comprising administering 3 mL-20 mL of the pharmaceutical composition of any one of [1]-[18] in the oral cavity and then getting the patient to swallow the pharmaceutical composition.

[22] The method of [21] wherein the mucosal disorder in the oral cavity and/or mucosal disorder in the pharynx is caused by radiation and chemotherapy, and the amount of the pharmaceutical composition administered in the oral cavity is 5 mL-10 mL.

[23] A method for preventing and/or treating mucosal disorder in the oral cavity, comprising repeating the operation of the method defined in [21] or [22] twice to six times a day.

[24] A method for preventing and/or treating mucosal disorder in the oral cavity and/or mucosal disorder in the pharynx caused by radiation and chemotherapy, comprising repeating the operation of the method defined in [21] or [22] twice to six times a day.

[25] A method for preventing and/or treating xerostomia and/or hyposalivation, comprising administering the pharmaceutical composition of any one of [1]-[18] in the oral cavity.

[26]. The pharmaceutical composition of [1]-[18], the above method for preparing the pharmaceutical composition, and the above method of the prevention and/or treatment, wherein rebamipide is a crystal form.

The pharmaceutical composition comprising rebamipide of the present invention, comprising

(a) rebamipide having a mean particle size of less than 500 nm (preferably, less than 300 nm),

(b) one or more dispersing agents,

(c) one or more viscosity enhancing agents which have no aggregative action for the rebamipide particles having a mean particle size of less than 500 nm (preferably, less than 300 nm),

(d) purified water,

(e) optionally one or more acids or one or more bases which might be necessary when preparing rebamipide having a mean particle size of less than 500 nm,

(f) optionally one or more pH adjusters

(g) optionally one or more antiseptic agents

(h) optionally one or more sweetening agents

(i) optionally one or more isotonic agents

(j) optionally one or more flavors

The mean particle size of rebamipide in the pharmaceutical composition of the present invention which is suitable for treating mucosal disorder in the oral cavity is preferably controlled to less than 500 nm. More preferably, the mean particle size is controlled to less than 300 nm, even more preferably less than 200 nm.

The term “mean particle size” means a mean volume diameter which can be measured by laser diffraction/scattering method. The particle size distribution can be measured by laser diffraction/scattering method, and the mean particle size can be obtained from the particle size distribution. The laser diffraction/scattering device used herein includes a laser diffraction particle size analyzer (SALD-3000J, SHIMADZU).

The rebamipide whose mean particle size is less than 500 nm in the pharmaceutical composition of the present invention can be prepared by any of various means. For example, it is possible to prepare a suspension comprising rebamipide having a mean particle size of less than 500 nm by suspending rebamipide in an aqueous solution containing a dispersing agent to give a suspension and milling it with a wet grinding media mill such as bead mill and ball mill. Such wet grinding media mill includes DYNO-MILL (Willy A Bachofen), ULTRA APEX MILL (KOTOBUKI INDUSTRIES CO., LTD.), Star mill (Ashizawa Finetech Ltd.), etc.

In addition, for example, rebamipide can be suspended in an aqueous solution containing a dispersing agent to obtain a suspension, and then the suspension can be milled with a high pressure wet disperser or a high pressure wet mill to prepare a suspension comprising rebamipide having a mean particle size of less than 500 nm. Such high pressure wet disperser and high pressure wet mill include Rannie-type or Gaulin-type high-pressure homogenizer (GEA Niro Soavi), Microfluidyzer (Micro fluidics), Star Burst System (SUGINO MACHINE LIMITED), Nanomizer (NANOMIZER Inc.) and Nano Jet Pal (JOKOH).

Alternatively, the pharmaceutical composition comprising rebamipide having a mean particle size of less than 500 nm can be prepared by mixing rebamipide with a dispersing agent, and/or sugars and other ingredient(s), milling the mixture with a dry mill such as a jet mill or a bead mill, and dispersing the milled mixture in an aqueous medium.

Preferably, the pharmaceutical composition comprising rebamipide having a mean particle size of less than 500 nm can be prepared by mixing at least one dispersing agent, an aqueous acid solution, an aqueous solution comprising a water-soluble rebamipide salt, and optional other ingredient(s) or solvent(s) to give an aqueous suspension comprising rebamipide.

The acid of the aqueous acid solution used herein includes, for example, a conventional acid such as hydrochloric acid, sulfuric acid, nitric acid, carbonic acid, phosphoric acid, and citric acid, and preferably hydrochloric acid.

The base to be added for preparing the aqueous solution comprising a water-soluble rebamipide salt includes, for example, a conventional base such as sodium hydroxide, potassium hydroxide, triethanolamine, tromethanol(tris(hydroxymethyl)aminomethane), meglumine, and diethanolamine, and preferably sodium hydroxide. The rebamipide can be used herein either as a salt or a free acid, but the rebamipide in the aqueous solution comprising a water-soluble rebamipide salt is accompanied with a base to be dissolved in the aqueous solution.

The dispersing agent used herein includes, for example, polyvinyl alcohol, hydroxypropylcellulose, hydroxyethyl cellulose, methylcellulose, hydroxypropylmethylcellulose, polyvinylpyrrolidone, polyethylene glycol (macrogol), polysorbate 80, carboxymethylcellulose sodium, polyacrylic acid, water-soluble chitosan, polyoxyethylene polyoxypropylene glycol, poly(oxyethylene) hydrogenated castor oil 40, poly(oxyethylene) hydrogenated castor oil 60, polyoxyl stearate 40, and gelatin; and one or more of the dispersing agents may be used.

Amongst them, hydroxypropylmethylcellulose, polyvinylpyrrolidone, polyoxyethylene polyoxypropylene glycol, and carboxymethylcellulose sodium are preferable.

The viscosity grade of the hydroxypropyl methylcellulose (2% aqueous solution) used herein is preferably 20 mPa·s or less, and the viscosity grade of the carboxymethylcellulose sodium (2% aqueous solution) used herein is preferably 50 mPa·s or less. The preferred polyoxyethylene polyoxypropylene glycol is polyoxyethylene (160) polyoxypropylene (30) glycol (Pluronic® F68).

Amongst the dispersing agents, polyvinylpyrrolidone is the most preferable. The mean molecular weight of the polyvinylpyrrolidone used herein is preferably 50,000 or less, and more preferably polyvinylpyrrolidone K25 or polyvinylpyrrolidone K30.

The concentration of the dispersing agent added in the pharmaceutical composition is preferably 0.1-10% (w/v), more preferably 0.3-5% (w/v), even more preferably 0.5-3% (w/v), and most preferably 1-2% (w/v).

The above method for mixing at least one dispersing agent, an aqueous acid solution, and an aqueous solution comprising a water-soluble rebamipide salt may be carried out by

(i) mixing an aqueous acid solution which comprises at least one dispersing agent, an aqueous solution comprising a water-soluble rebamipide salt, and optional other ingredient(s) or solvent(s),

(ii) mixing an aqueous acid solution, an aqueous solution comprising a water-soluble rebamipide salt which comprises at least one dispersing agent, and optional other ingredient(s) or solvent(s), or

(iii) mixing an aqueous acid solution which comprises at least one dispersing agent, an aqueous solution comprising a water-soluble rebamipide salt which comprises the same at least one dispersing agent, and optional other ingredient(s) or solvent(s).

The method for mixing the solutions is not limited to specific ones, but preferably the solutions are mixed while stirring with a conventional stirrer such as a disperser, homomixer, and homogenizer, and charging a shearing force in the dispersing device. In addition, the ultrasonication may be used in mixing them.

As mentioned above, to the aqueous suspension comprising rebamipide crystal prepared by mixing at least one dispersing agent, an aqueous acid solution, an aqueous solution comprising a water-soluble rebamipide salt, and optional other ingredient(s) or solvent(s) was added a base to adjust the pH to 3-7, and then preferably the aqueous suspension is stirred/dispersed and/or dialyzed. The base used herein may be the same as the aforementioned base.

The stirring and dispersing machines used herein can be selected from various conventional stirring and dispersing machines used for pharmaceutical formulation such as a disperser, a homomixer, and a homogenizer, preferably a stirring and dispersing machine which makes “aggregated particles in the liquid” effectively-dispersed. The preferred examples include a rotary homogenizer such as ROBOMICS® (PRIMIX Corporation) and CLEARMIX® (M Technique Co., Ltd.), a wet-type jet mill and a high-pressure homogenizer. In particular, in using CLEARMIX® W-MOTION (M Technique Co., Ltd.) wherein a screen and a rotor are counter-rotated at high speed to give a strong liquid-liquid shearing force, the dispersibility of the primary particles in the aqueous suspension comprising rebamipide prepared above can be enhanced.

The present inventors have found that it is possible to prepare a suspension comprising rebamipide wherein the rebamipide particles are not aggregated even when stored for a long time, by dialyzing the aqueous suspension comprising rebamipide crystallized as above, and making the mean particle size of the rebamipide crystal adjusted to less than 500 nm. The dialyzing system used herein can be chosen from conventional dialyzing systems such as Pellicon® (MILLIPORE), ProStack® (MILLIPORE), and Sartocon® (SARTORIUS K.K.). In case of low pH, the aqueous suspension comprising rebamipide to be dialyzed has a bad flow to a dialysis membrane due to aggregation, while in case of high pH, the content of rebamipide is decreased because rebamipide is dissolved. Thus, the dialysis should be carried out at pH of 3-7, preferably 4-7, more preferably 5-7. The dialysis process and the dispersion/stir process can be carried out separately. Or, the both processes can be done in combination, i.e., the dispersion/stir process can be done after the dialysis process is done, or the dialysis process can be done after the dispersion/stir process is done.

The shape of rebamipide prepared via mixing at least one dispersing agent, an aqueous acid solution, an aqueous solution comprising a water-soluble rebamipide salt, and optional other ingredient(s) or solvent(s) is a uniform needle crystal having the longest diameter of less than 1000 nm and the shortest diameter of less than 60 nm, provided that the longest diameter/the shortest diameter is more than 3.

When polyvinylpyrrolidone is used as a dispersing agent, it is possible to obtain a suspension comprising a uniform needle crystal having the longest diameter of less than 500 nm and the shortest diameter of less than 60 nm, preferably the longest diameter of less than 300 nm and the shortest diameter of less than 50 nm, provided that the longest diameter/the shortest diameter is more than 3; even more preferably a suspension comprising a uniform needle crystal having the longest diameter of about 200 nm and the shortest diameter of about 40 nm, provided that the longest diameter/the shortest diameter is about 5, through the above method.

The pharmaceutical composition of the present invention comprises a viscosity enhancing agent. The preferred viscosity enhancing agent has no aggregative action for the rebamipide particles having a mean particle size of less than 500 nm. The term “no aggregative action” means that the rebamipide keeps its mean particle size less than 500 nm when the viscosity enhancing agent is added to a suspension comprising rebamipide having a mean particle size of less than 500 nm. Preferably, it means that the rebamipide keeps its mean particle size less than 300 nm when the viscosity enhancing agent is added to a suspension comprising rebamipide having a mean particle size of less than 300 nm. Furthermore, it is necessary to keep the mean particle size of the rebamipide in the suspension stored for at least one year less than 500 nm in order to secure the pharmaceutical market.

A suspension comprising rebamipide having a mean particle size of less than 500 nm is apt to be aggregated by adding a viscosity enhancing agent, and a viscosity enhancing agent that does not cause the aggregative action is rare. Carrageenan (carrageenin), guar gum, gellan gum, hyaluronic acid, carboxy vinyl polymer, sodium chondroitin sulfate, and sodium alginate which are generally used as a viscosity enhancing agent cannot be used herein because they can make the above-prepared rebamipide particles having a mean particle size of less than 500 nm aggregated.

The viscosity enhancing agent used herein includes hydroxypropylcellulose, polyvinyl alcohol, carboxymethylcellulose sodium, polyvinylpyrrolidone K90, and pullulan.

When hydroxypropylmethylcellulose is used as a dispersing agent, the preferred viscosity enhancing agent is hydroxypropylcellulose, pullulan, etc. When polyoxyethylene polyoxypropylene glycol is used as a dispersing agent, the preferred viscosity enhancing agent is polyvinyl alcohol, pullulan, etc. When carboxymethylcellulose sodium is used as a dispersing agent, the preferred viscosity enhancing agent is a high molecular weight (high viscosity grade) carboxymethylcellulose sodium, hydroxypropylcellulose, polyvinylpyrrolidone K90 and pullulan. When polyvinylpyrrolidone K25 or polyvinylpyrrolidone K30 is used as a dispersing agent, the preferred viscosity enhancing agent is polyvinyl alcohol, polyvinylpyrrolidone K90, pullulan, etc.

The preferred concentration of a viscosity enhancing agent in the present pharmaceutical composition is 5 mg/mL-150 mg/mL, more preferably 10 mg/mL-60 mg/mL, and even more preferably 15 mg/mL-40 mg/mL.

The present pharmaceutical composition containing a viscosity enhancing agent is a viscous liquid preparation, and the viscosity of the liquid preparation is 10 mPa·s-500 mPa·s, the preferred viscosity of the liquid preparation is 20 mPa·s-300 mPa·s, the most preferred viscosity thereof is 30 mPa·s-200 mPa·s. The viscosity shown herein is measured by Viscosity Determination defined in the Japanese Pharmacopoeia, for example, using a cone-flat plate-type rotational viscometer (cone plate type viscometer) at 25° C.

The present inventors have extensively studied, and have found that the addition of hydroxypropylcellulose and/or pullulan as a viscosity enhancing agent in case of using hydroxypropylmethylcellulose as a dispersing agent can prevent the aggregation of rebamipide and can bring in the enhanced viscosity. And, it has been found that the addition of polyvinyl alcohol and/or pullulan as a viscosity enhancing agent in case of using polyoxyethylene polyoxypropylene glycol as a dispersing agent can prevent the aggregation of rebamipide and can bring in the enhanced viscosity. In addition, it has been found that the addition of a high molecular weight (high viscosity grade) carboxymethylcellulose sodium, hydroxypropylcellulose, polyvinylpyrrolidone K90 and/or pullulan as a viscosity enhancing agent in case of using carboxymethylcellulose sodium as a dispersing agent can prevent the aggregation of rebamipide and can bring in the enhanced viscosity. Furthermore, it has been found that the addition of polyvinyl alcohol, polyvinylpyrrolidone K90 and/or pullulan as a viscosity enhancing agent in case of using polyvinylpyrrolidone K25 or polyvinylpyrrolidone K30 as a dispersing agent can prevent the aggregation of rebamipide and can bring in the enhanced viscosity. It was unexpected that the type of viscosity enhancing agent which can prevent the aggregation of rebamipide is specifically different depending on the type of dispersing agent for rebamipide.

In particular, the addition of a combination of polyvinylpyrrolidone K90 and pullulan as a viscosity enhancing agent in case of using polyvinylpyrrolidone K25 or polyvinylpyrrolidone K30 as a dispersing agent made it possible to prevent the aggregation of rebamipide and bring in a preferred viscosity of the liquid preparation. Further surprisingly, when polyvinylpyrrolidone K90 and pullulan are added to 10 mg/mL-40 mg/mL aqueous suspension comprising rebamipide wherein the mean particle size is more than 1 μm, the viscosity was not enhanced, compared with the solution comprising only polyvinylpyrrolidone K90 and pullulan. While, when polyvinylpyrrolidone K90 and pullulan are added to 10 mg/mL-40 mg/mL aqueous suspension comprising rebamipide wherein the mean particle size is less than 500 nm, the viscosity was remarkedly enhanced and it became possible to bring in a preferred viscosity of the liquid preparation. It was quite unexpected. The preferred ranges of polyvinylpyrrolidone K90 and pullulan which are added as a viscosity enhancing agent are a combination of 5 mg/mL-50 mg/mL and 10 mg/mL-100 mg/mL, respectively. The more preferred ranges of the added polyvinylpyrrolidone K90 and pullulan are a combination of 5 mg/mL-30 mg/mL and 10 mg/mL-30 mg/mL, respectively. The most preferred ranges are both 10 mg/mL-20 mg/mL polyvinylpyrrolidone K90 and 20 mg/mL pullulan, in which there arises neither precipitation nor aggregation of rebamipide particles at room temperature, and the suitable viscosity of the liquid preparation is obtained.

The present inventors have extensively studied and then found that a pharmaceutical composition as an aqueous suspension comprising 10 mg/mL-50 mg/mL rebamipide having a mean particle size of less than 500 nm as an active ingredient, at least one dispersing agent, and at least one viscosity enhancing agent, wherein the viscosity enhancing agent has no aggregative action for the rebamipide particles having a mean particle size of less than 500 nm and the viscosity of the liquid preparation is in a range of 10 mPa·s-500 mPa·s; preferably a pharmaceutical composition as an aqueous suspension comprising 20 mg/mL-40 mg/mL rebamipide having a mean particle size of less than 300 nm as an active ingredient, at least one dispersing agent, and at least one viscosity enhancing agent, wherein the viscosity enhancing agent has no aggregative action for the rebamipide particles having a mean particle size of less than 300 nm and the viscosity of the liquid preparation is in a range of 20 mPa·s-300 mPa·s, has a significant healing effect for oral ulcer in a stomatitis rat model. This effect is not found in the conventional suspension comprising 1 mg/mL or 2 mg/mL rebamipide having a mean particle size of 1 μm or more, thus it is a quite surprise. As see in Comparative example, the suspension comprising rebamipide having a mean particle size of 1 μm or more had no healing effect for oral ulcer even when the concentration is 20 mg/mL. While, the present suspension rebamipide having a mean particle size of less than 500 nm had significant healing effect for oral ulcer in a stomatitis rat model when the concentration is 20 mg/mL. And, in the present pharmaceutical composition, there is no aggregative action for the rebamipide particles, hence the present pharmaceutical composition has an industrial merit to keep the stability for distribution in the pharmaceutical market.

The present invention is directed to a suspension comprising rebamipide particles wherein the rebamipide particles are not aggregated, which has suitable viscosity and suitable fluidity and does not include the suspensible hydrogel disclosed in WO 2007/132907. The rebamipide having a mean particle size of less than 500 nm in a suspensible hydrogel has the interaction between the rebamipide crystals (aggregation), thereby a hydrogel thereof having the thixotropic nature is supposed to be produced. Such hydrogel is not suitable for the present use for treating stomatitis because the particles are aggregated.

In addition, the pharmaceutical composition of the present invention can further comprise some ingredients which are generally used in oral liquid agents such as a preserving agent (antiseptic agent), an isotonic agent, a sweetening agent, a flavor, and a pH adjuster, if necessary; and it is possible to prepare a useful formulation with the pharmaceutical composition.

The pharmaceutical composition of the present invention may further comprise a preserving agent (antiseptic agent) in order to prevent the bacterial contamination in the invention product in the pharmaceutical market. The preserving agent (antiseptic agent) used herein includes, for example, a quaternary ammonium salt such as benzalkonium chloride, and benzethonium chloride; a cationic compound such as chlorhexidine gluconate; a parahydroxybenzoate such as methyl parahydroxybenzoate, ethyl parahydroxybenzoate, and propyl parahydroxybenzoate; an alcoholic compound such as chlorobutanol, and benzyl alcohol; sodium dehydroacetate; and thimerosal, and such preserving agent preferably does not cause the aggregation of the rebamipide particles. The present inventors have extensively studied and then found that parahydroxybenzoates are preferable as a preserving agent which does not cause the aggregation of the rebamipide particles, in particular, methyl parahydroxybenzoate and ethyl parahydroxybenzoate are the most preferable. Either methyl parahydroxybenzoate or ethyl parahydroxybenzoate can be used alone, but a combination thereof is more preferable. The preferred amount of methyl parahydroxybenzoate is 0.5 mg/mL-2 mg/mL, and the preferred amount of ethyl parahydroxybenzoate is 0.1 mg/mL-0.8 mg/mL.

The pharmaceutical composition of the present invention can comprise an isotonic agent in order to prevent the irritation for stomatitis. The preferred isotonic agent used herein is nonionic isotonic agent. The nonionic isotonic agent used herein includes a general nonionic isotonic agent for medical use such as mannitol, glycerin, sorbitol, glucose, xylitol, trehalose, maltose, and maltitol, which is preferably added to the composition in an amount to make the composition isotonic.

The present pharmaceutical composition has a bitter taste because it comprises rebamipide as an active ingredient which is known as a bitter material. Thus, it is possible to add a sweetening agent in order to lower the bitter taste. The sweetening agent used herein includes aspartame, sucralose, acesulfame K, saccharin, saccharin sodium, stevia, and thaumatin. The present inventors have extensively studied and then found that stevia is a preferred sweetening agent which does not cause the aggregation of the rebamipide particles and can lower the bitter taste to have the present composition administered in the oral cavity. The preferred amount of stevia is 0.5 mg/mL-1 mg/mL.

The pharmaceutical composition of the present invention can further comprise a flavor in order to lower the bitter taste of rebamipide. The flavor used herein includes, for example, a generally available flavor for medical use such as orange flavor, orange essence, grapefruit flavor, strawberry flavor, mint flavor, cocoa flavor, coffee flavor, and chocolate flavor. The preferred amount of the flavor is 0.5 mg/mL-1 mg/mL.

To the aqueous suspension comprising rebamipide can be added a pH adjuster such as an acid (e.g. hydrochloric acid, sulfuric acid, nitric acid, carbonic acid, phosphoric acid, and citric acid) and a base (e.g. sodium hydroxide, potassium hydroxide, triethanolamine, tromethanol [tris(hydroxymethyl)aminomethane], meglumine, and diethanolamine to adjust the pH to 5-7, preferably 5.5-6.5 in which there is little irritation in the oral cavity.

In addition, the pharmaceutical composition of the present invention can comprise a buffer, a stabilizer, etc.

The buffer used herein includes, for example, acetic acid and an acetate such as sodium acetate; citric acid or a salt thereof; a phosphate such as sodium dihydrogen phosphate, disodium hydrogen phosphate, potassium dihydrogen phosphate, and dipotassium hydrogen phosphate; epsilon-aminocapronic acid; an amino acid salt such as sodium glutamate, and boric acid and a salt thereof.

The stabilizer includes, for example, ascorbic acid and a salt thereof, tocopherol, sodium thiosulfate, sodium bisulfite, and disodium edetate.

The method for preparing the pharmaceutical composition of the present invention can comprise

adding a viscosity enhancing agent to an aqueous suspension comprising rebamipide having a mean particle size of less than 500 nm and a dispersing agent, as mentioned above,

optionally adding various ingredients such as preserving agent (antiseptic agent), isotonic agent, sweetening agent, and flavor, if necessary, and

adjusting the pH to 5-7, preferably 5.5-6.5 with a pH adjuster.

The most preferred method for preparing the pharmaceutical composition of the present invention comprises

mixing at least one dispersing agent, an aqueous acid solution, an aqueous solution comprising a water-soluble rebamipide salt, and optional other ingredient(s) or solvent(s) to prepare an aqueous suspension comprising rebamipide crystal,

adding a base thereto to adjust pH of the aqueous suspension to 3-7,

dispersing and/or dialyzing the aqueous suspension,

adjusting pH of the aqueous suspension to 5-7, and then

adding a viscosity enhancing agent thereto and optionally adding a preserving agent (antiseptic agent), an isotonic agent, a sweetening agent, and a flavor thereto.

As mentioned above, the pharmaceutical composition of the present invention can comprise

a parahydroxybenzoate as a preserving agent (antiseptic agent), a non-ionic isotonic agent as an isotonic agent,

stevia as a sweetening agent, and a flavor, a pH adjuster,

preferably, 0.5 mg/mL-2 mg/mL methyl parahydroxybenzoate, 0.1 mg/mL-0.8 mg/mL ethyl parahydroxybenzoate, a non-ionic isotonic agent whose amount is adjusted to make the composition isotonic, 0.5 mg/mL-1 mg/mL stevia, and a flavor, and a pH adjuster which is added to adjust the pH to 5.5-6.5,

which does not make rebamipide having a mean particle size of less than 500 nm aggregated, prevents the bacterial growth in the invention product in the pharmaceutical market, lowers the bitter taste of rebamipide to have the present composition administered in the oral cavity, and prevents the irritation in the oral cavity. At the above point, the present invention is very useful in the industrial utility.

The use of the present pharmaceutical composition includes the prevention and/or treatment of mucosal disorder in the oral cavity and/or mucosal disorder in the pharynx, preferably the prevention and/or treatment of mucosal disorder in the oral cavity caused by radiation and chemotherapy in the cancer therapy. More preferably, it includes the prevention and/or treatment of mucosal disorder in the oral cavity caused by radiation in treating head and neck cancer. Furthermore, it is also useful for preventing or treating xerostomia and/or hyposalivation.

As a method of using the present pharmaceutical composition, it is useful for preventing and/or treating mucosal disorder in the oral cavity to administer the present composition in the oral cavity (gargle) and preferably swallow the pharmaceutical composition (swish and swallow). The dosing volume is 3 mL-20 mL, preferably 5 mL-10 mL, more preferably 7 mL-8 mL, per once. The above gargle or a liquid preparation for swish and swallow is preferably repeated twice to 6 times, preferably 4 times to 6 times, more preferably 4 times per day. The hitherto known suspension formulation comprising rebamipide is a suspension formulation comprising 1-2 mg/mL rebamipide having a mean particle size of 1 μm or more. As shown in Comparative example 1, however, such formulation has no healing effect for oral ulcer in a stomatitis rat model even when the concentration is 20 mg/mL.

While, the aqueous suspension of the present invention comprising rebamipide having a mean particle size of less than 500 nm (preferably, 300 nm) wherein the viscosity of the liquid preparation is in a range of 10 mPa·s-500 mPa·s (preferably, 20 mPa·s-300 mPa·s) had significant healing effect for oral ulcer in a stomatitis rat model when the concentration is 20 mg/mL in which a hitherto known formulation (e.g. Comparative example 1) or a formulation out of the present invention (e.g. Comparative examples 2 and 3) had no effect.

The pharmaceutical composition of the present invention can be used as gargle or a liquid preparation for swish and swallow. In case of the prevention and treatment of mucosal disorder in the oral cavity caused by radiation in treating head and neck cancer, the liquid preparation for swish and swallow is more preferably because the disorder can be accompanied with pharyngitis and esophagitis. In case of the liquid preparation for swish and swallow, the pharmaceutical composition preferably has a potent effect thereof to make the dose lowered in consideration of systemic side effects. The pharmaceutical composition of the present invention is also useful at this point.

### Effect of the Invention

The pharmaceutical composition of the present invention which has a significant healing effect for oral ulcer in a stomatitis rat model, is very useful as a medicament for treating stomatitis which has been a problem in cancer therapy, and also meaningful in the industrial utility. In addition, it has been found that the composition of the present invention can inhibit oral ulcer in a radiated rat model. Thus, it is suggested that the present pharmaceutical composition has a healing effect for oral ulcer as well as a potent preventing effect for mucosal disorder in the oral cavity (stomatitis) caused by radiation which has been a problem in treating head and neck cancer. Thereby, the present invention can make it possible to continue a clinical radiotherapy, and it is suggested that the present invention can raise the score of the treatment for head and neck cancer.

In addition, the present invention can keep the stability for distribution in the pharmaceutical market without aggregating rebamipide having a mean particle size of less than 500 nm. And, the present invention can prevents the bacterial growth in the invention product in the pharmaceutical market without aggregating rebamipide having a mean particle size of less than 500 nm. Furthermore, an aqueous solution in which rebamipide is simply dissolved has a terribly bitter taste and it is hard to be administered; while the present invention has no problem to be administered, which is a liquid preparation for oral administration comprising rebamipide having bitter taste, and the irritation in the oral cavity can be prevented. As mentioned above, the present invention has very useful properties as a medicament for treating stomatitis which has been a problem in cancer therapy, and then it is expected to contribute to the cancer therapy. Thus, the present invention is a quite useful pharmaceutical composition in the medical/industrial field.

### DESCRIPTION OF EMBODIMENTS

The present invention is illustrated in more detail by the following examples, but should not be construed to be limited thereto.

### EXAMPLE

### Example 1

20 g of carboxymethylcellulose sodium (CMCNa) (7L2P, Ashland) was dissolved in about 400 g of purified water. Thereto, 28.4 g of concentrated hydrochloric acid, and further purified water were added to prepare 550 g of an aqueous solution of carboxymethylcellulose sodium (7L2P)-hydrochloric acid. Separately, 17.6 g of sodium hydroxide was added to about 2600 g of purified water to prepare an aqueous sodium hydroxide. 81.6 g of rebamipide (Otsuka Pharmaceutical Co., Ltd.) was dissolved in the aqueous sodium hydroxide while warming the solution, and then purified water was added thereto to adjust the total weight to 2940 g. From the prepared sodium hydroxide-rebamipide solution, 1470 g thereof was taken out for the next step.

To the aqueous solution of carboxymethylcellulose sodium-hydrochloric acid, which was stirred at 5500 rpm with a disperser (ROBOMIX®, PRIMIX Corporation), cooled in ice bath, the above sodium hydroxide-rebamipide solution whose temperature was maintained at about 50° C. was gradually added to precipitate a rebamipide crystal. After all the sodium hydroxide-rebamipide solution was added thereto, the liquid preparation was stirred for 20 minutes. After the liquid preparation was allowed to stand overnight, 5 N aqueous sodium hydroxide was added to the liquid preparation to adjust pH of the liquid preparation to about 5.8.

The resulting aqueous suspension of rebamipide was dispersed for 40 minutes with CLEARMIX W-MOTION (M Technique Co., Ltd.) wherein the rotor was set at about 18000 rpm, and the screen was set at about 16000 rpm. The liquid preparation was concentrated/desalted with a dialyzing system (Pellicon® 2 Mini, MILLIPORE).

After measuring the concentration of rebamipide in the concentrated/desalted sample, carboxymethylcellulose sodium (CELLOGEN PRS, DAI-ICHI KOGYO SEIYAKU CO., LTD.), D-sorbitol (Wako Pure Chemical Industries, Ltd.) and purified water were added to the sample to prepare 2% rebamipide suspension so that the concentrations of carboxymethylcellulose sodium (CELLOGEN PRS) and D-sorbitol would be 3% and 4%, respectively.

| Ingredient | | Weight per 1 mL |
|---|---|---|
| rebamipide | active ingredient | 20 mg |
| carboxymethylcellulose | dispersing agent | 10 mg |
| sodium (7L2P) | | |
| carboxymethylcellulose | viscosity | 30 mg |
| sodium (CELLOGEN PRS) | enhancing agent | |
| D-sorbitol | isotonic agent | 40 mg |
| purified water | solvent | adjust the whole |
| | | volume to 1 mL |

The viscosity of the suspension measured with a rotatory viscometer (RC-100A, TOKI SANGYO CO., LTD.) was 33 mPa·s. The mean particle size was measured by dispersing the rebamipide suspension in water, with a laser diffraction particle size analyzer (SALD-3000J, Shimadzu Corporation). The mean particle size was 0.18 μm (without ultrasonic irradiation, refractive index: 1.70-0.201).

### Comparative Example 1

According to the amounts defined the following table, carboxymethylcellulose sodium (Wako Pure Chemical Industries, Ltd.) and D-sorbitol (Wako Pure Chemical Industries, Ltd.) were dissolved in 100 mL of purified water, and pH of the solution was adjusted to 6.0-6.2. Then, rebamipide powder (Otsuka Pharmaceutical Co., Ltd.) was added to the solution to prepare 2% rebamipide suspension.

| Ingredient | | Weight per 1 mL |
|---|---|---|
| rebamipide | active ingredient | 20 mg |
| carboxymethyl- | dispersing agent | 5 mg |
| cellulose | | |
| sodium | | |
| D-sorbitol | isotonic agent | 40 mg |
| purified water | solvent | adjust the whole |
| | | volume to 1 mL |

The viscosity of the suspension measured with a rotatory viscometer (RC-100A, TOKI SANGYO CO., LTD.) was 12 mPa·s. The mean particle size was measured by dispersing the rebamipide suspension in water, with a laser diffraction particle size analyzer (SALD-3000J, Shimadzu Corporation). The mean particle size was 13.9 μm (without ultrasonic irradiation, refractive index: 2.00-0.201).

### Test 1

Oral ulcer was induced by cautery method as mentioned below. In detail, the normally-bred rat was anesthetized by inhalation of isoflurane. Under dorsal position, upper and lower jaws of rat were opened with a rib retractor to obtain a viewing field, and the center of left-inside buccal mucosa was cauterized in a circle (diameter: 3-4 mm) by contacting with a monopolar tip which has a diameter of 2 mm, for about 10-20 seconds (power output: 20) to induce oral ulcer. After the cautery treatment, the rat was put back in a breeding cage and was waken therein naturally.

The day on which the oral ulcer was induced was defined as the starting day (Day 0). Two days after the induction of the oral ulcer (Day 2), the treated rats were divided in predetermined groups by stratified randomization based on the body weight. From the third day after the induction of the oral ulcer by cautery method (Day 3), 2% rebamipide suspension of Example 1, 2% rebamipide suspension of Comparative example 1, and each solvent thereof (i.e., each solvent obtained by excluding rebamipide from the Example and the Comparative example) were intra-orally administered to the rats in a volume of 0.5 mL/kg, four times a day (about 8:00, 11:00, 14:00 and 17:00) for 5 days. The rats were anesthetized by inhalation of isoflurane and placed in the left lateral decubitus position, and then administered each test sample into the left oral cavity having oral ulcer after making the mouth opened with forceps or a rib retractor.

The area of the oral ulcer was measured on Day 8. The area of the oral ulcer in the group treated with 2% rebamipide suspension of Example 1 was significantly decreased, compared with the group treated with the solvent (n=6, p<0.01, t-test). The decrease ratio of the ulcer area in the group treated with the rebamipide suspension of Example 1 was 20.1% as compared to the ulcer area in the group treated with the solvent.

On the other hand, the ulcer area in the group treated with the 2% rebamipide suspension of Comparative example 1 was not significantly decreased for the group treated with the solvent (n=6, n.s., t-test). The decrease ratio of the ulcer area in the group treated with the rebamipide suspension of Comparative example 1 was 8.7% as compared to the ulcer area in the group treated with the solvent.

### Example 2

40 g of hydroxypropylmethylcellulose (HPMC) (TC-5E, Shin-Etsu Chemical Co., Ltd.) was dissolved in about 400 g of purified water. Thereto, 28.4 g of concentrated hydrochloric acid, and further purified water were added to prepare 550 g of an aqueous solution of HPMC (TC-5E)-hydrochloric acid. Separately, 17.6 g of sodium hydroxide was added to about 2600 g of purified water to prepare an aqueous sodium hydroxide. 81.6 g of rebamipide (Otsuka Pharmaceutical Co., Ltd.) was dissolved in the aqueous sodium hydroxide while warming the solution, and then purified water was added thereto to adjust the total weight to 2940 g. From the prepared sodium hydroxide-rebamipide solution, 1470 g thereof was taken out for the next step.

To the aqueous solution of HPMC (TC-5E)-hydrochloric acid, which was stirred at 5500 rpm with a disperser (ROBOMIX®, PRIMIX Corporation), cooled in ice bath, the above sodium hydroxide-rebamipide solution whose temperature was maintained at about 50° C. was gradually added to precipitate a rebamipide crystal. After all the sodium hydroxide-rebamipide solution was added thereto, the liquid preparation was stirred for 20 minutes. After the liquid preparation was allowed to stand overnight, 5 N aqueous sodium hydroxide was added to the liquid preparation to adjust pH of the liquid preparation to about 5.8.

The resulting aqueous suspension of rebamipide was dispersed for 40 minutes with CLEARMIX W-MOTION (M Technique Co., Ltd.) wherein the rotor was set at about 18000 rpm, and the screen was set at about 16000 rpm. The liquid preparation was concentrated/desalted with a dialyzing system (Pellicon® 2 Mini, MILLIPORE).

After measuring the concentration of rebamipide in the concentrated/desalted sample, hydroxypropylcellulose (HPC-L, NIPPON SODA CO., LTD.), D-sorbitol (Wako Pure Chemical Industries, Ltd.) and purified water were added to the sample to prepare 2% rebamipide suspension so that the concentrations of hydroxypropylcellulose and D-sorbitol would be 2% and 4%, respectively.

| Ingredient | | Weight per 1 mL |
|---|---|---|
| rebamipide | active ingredient | 20 mg |
| HPMC (TC-5E) | dispersing agent | 20 mg |
| HPC (HPC-L) | viscosity enhancing | 20 mg |
| | agent | |
| D-sorbitol | isotonic agent | 40 mg |
| purified water | solvent | adjust the whole |
| | | volume to 1 mL |

The viscosity of the suspension measured with a rotatory viscometer (RC-100A, TOKI SANGYO CO., LTD.) was 42 mPa·s. The mean particle size was measured by dispersing the rebamipide suspension in water, with a laser diffraction particle size analyzer (SALD-3000J, Shimadzu Corporation). The mean particle size was 0.17 μm (without ultrasonic irradiation, refractive index: 1.70-0.20 i).

### Comparative Example 2

After measuring the concentration of rebamipide in the concentrated/desalted sample prepared in Example 2, D-sorbitol and purified water were added to the sample to prepare 2% rebamipide suspension so that the concentrations of D-sorbitol would be 4%.

| Ingredient | | Weight per 1 mL |
|---|---|---|
| rebamipide | active ingredient | 20 mg |
| HPMC (TC-5E) | dispersing agent | 20 mg |
| D-sorbitol | isotonic agent | 40 mg |
| purified water | solvent | adjust the whole |
| | | volume to 1 mL |

The viscosity of the suspension measured with rotatory viscometer (RC-100A, TOKI SANGYO CO., LTD.) was 8 mPa·s. The mean particle size was measured by dispersing the rebamipide suspension in water, with a laser diffraction particle size analyzer (SALD-3000J, Shimadzu Corporation). The mean particle size was 0.08 μm (without ultrasonic irradiation, refractive index: 1.70-0.20 i).

### Test 2

As mentioned in Test 1, oral ulcer was induced to the rats, and then the rats were divided in predetermined groups. From the third day after the induction of oral ulcer by cautery method (Day 3), 2% rebamipide suspension of Example 2, 2% rebamipide suspension of Comparative example 2, and each solvent thereof (i.e., each solvent obtained by excluding rebamipide from the Example and the Comparative example) were intra-orally administered to the rats in a volume of 0.5 mL/kg, four times a day (about at 8:00, 11:00, 14:00 and 17:00) for 5 days. The rats were anesthetized by inhalation of isoflurane and placed in the left lateral decubitus position, and then administered each test sample into the left oral cavity having oral ulcer after making the mouth opened with forceps or a rib retractor.

The area of the oral ulcer was measured on Day 8. The area of the oral ulcer in the group treated with 2% rebamipide suspension of Example 2 was significantly decreased, compared with the group treated with the solvent (n=6, p<0.05, t-test). The decrease ratio of the ulcer area in the group treated with the rebamipide suspension of Example 2 was 18.1% as compared to the ulcer area in the group treated with the solvent.

On the other hand, the ulcer area in the group treated with the 2% rebamipide suspension of Comparative example 2 was not significantly decreased for the group treated with the solvent (n=6, n.s., t-test). The decrease ratio of the ulcer area in the group treated with the rebamipide suspension of Comparative example 2 was 10.2% as compared to the ulcer area in the group treated with the solvent.

### Example 3

40 g of polyvinylpyrrolidone K25 (PVPK25) (BASF) was dissolved in about 400 g of purified water. Thereto, 28.4 g of concentrated hydrochloric acid, and further purified water were added to prepare 550 g of an aqueous solution of PVPK25-hydrochloric acid. Separately, 17.6 g of sodium hydroxide was added to about 2600 g of purified water to prepare an aqueous sodium hydroxide. 81.6 g of rebamipide (Otsuka Pharmaceutical Co., Ltd.) was dissolved in the aqueous sodium hydroxide while warming the solution, and then purified water was added thereto to adjust the total weight to 2940 g. From the prepared sodium hydroxide-rebamipide solution, 1470 g thereof was taken out for the next step.

To the aqueous solution of PVPK25-hydrochloric acid, which was stirred at 5500 rpm with a disperser (ROBOMIX®, PRIMIX Corporation), cooled in ice bath, the above sodium hydroxide-rebamipide solution whose temperature was maintained at about 50° C. was gradually added to precipitate a rebamipide crystal. After all the sodium hydroxide-rebamipide solution was added thereto, the liquid preparation was stirred for 20 minutes. After the liquid preparation was allowed to stand overnight, 5 N aqueous sodium hydroxide was added to the liquid preparation to adjust pH of the liquid preparation to about 5.8.

The resulting aqueous suspension of rebamipide was dispersed for 40 minutes with CLEARMIX W-MOTION (M Technique Co., Ltd.) wherein the rotor was set at about 18000 rpm, and the screen was set at about 16000 rpm. The liquid preparation was concentrated/desalted with a dialyzing system (Pellicon® 2 Mini, MILLIPORE).

After measuring the concentration of rebamipide in the concentrated/desalted sample, polyvinylpyrrolidone K90 (PVPK90) (BASF), stevia (Steviron® C, Morita Kagaku Kogyo Co., Ltd.), D-sorbitol (Wako Pure Chemical Industries, Ltd.), and purified water were added to the sample to prepare 2% rebamipide suspension so that the concentrations of polyvinylpyrrolidone K90, stevia, and D-sorbitol would be 3%, 0.05%, and 4%, respectively.

| Ingredient | | Weight per 1 mL |
|---|---|---|
| rebamipide | active ingredient | 20 mg |
| polyvinyl- | dispersing agent | 20 mg |
| pyrrolidone | | |
| K25 | | |
| polyvinyl- | viscosity enhancing | 30 mg |
| pyrrolidone | agent | |
| K90 | | |
| D-sorbitol | isotonic agent | 40 mg |
| stevia | sweetening agent | 0.5 mg |
| purified water | solvent | adjust the whole |
| | | volume to 1 mL |

The viscosity of the suspension measured with a rotatory viscometer (RC-100A, TOKI SANGYO CO., LTD.) was 25 mPa·s. The mean particle size was measured by dispersing the rebamipide suspension in water, with a laser diffraction particle size analyzer (SALD-3000J, Shimadzu Corporation). The mean particle size was 0.09 μm (without ultrasonic irradiation, refractive index: 1.70-0.20 i).

### Example 4

20 g of polyvinylpyrrolidone K30 (PVPK30) (BASF) was dissolved in about 400 g of purified water. Thereto, 28.4 g of concentrated hydrochloric acid, and further purified water were added to prepare 550 g of an aqueous solution of PVPK30-hydrochloric acid. Separately, 17.6 g of sodium hydroxide was added to about 2600 g of purified water to prepare an aqueous sodium hydroxide. 81.6 g of rebamipide (Otsuka Pharmaceutical Co., Ltd.) was dissolved in the aqueous sodium hydroxide while warming the solution, and then purified water was added thereto to adjust the total weight to 2940 g. From the prepared sodium hydroxide-rebamipide solution, 1470 g thereof was taken out for the next step.

To the aqueous solution of PVPK30-hydrochloric acid, which was stirred at 3000 rpm with a disperser (ROBOMIX®, PRIMIX Corporation), cooled in ice bath, the above sodium hydroxide-rebamipide solution whose temperature was maintained at about 50° C. was gradually added to precipitate a rebamipide crystal. After all the sodium hydroxide-rebamipide solution was added thereto, the liquid preparation was stirred for 30 minutes. After the liquid preparation was allowed to stand overnight, 5 N aqueous sodium hydroxide was added to the liquid preparation to adjust pH of the liquid preparation to about 5.8.

The resulting aqueous suspension of rebamipide was dispersed for 40 minutes with CLEARMIX W-MOTION (M Technique Co., Ltd.) wherein the rotor was set at about 18000 rpm, and the screen was set at about 16000 rpm. The liquid preparation was concentrated/desalted with a dialyzing system (Pellicon® 2 Mini, MILLIPORE).

After measuring the concentration of rebamipide in the concentrated/desalted sample, pullulan, stevia (Steviron® C, Morita Kagaku Kogyo Co., Ltd.), D-sorbitol (Wako Pure Chemical Industries, Ltd.), methylparaben, and purified water were added to the sample to prepare 2% rebamipide suspension so that the concentrations of pullulan, stevia, D-sorbitol, and methylparaben would be 5%, 0.05%, 4% and 0.1%, respectively.

| Ingredient | | Weight per 1 mL |
|---|---|---|
| rebamipide | active ingredient | 20 mg |
| polyvinyl- | dispersing agent | 10 mg |
| pyrrolidone | | |
| K30 | | |
| pullulan | viscosity enhancing | 50 mg |
| | agent | |
| D-sorbitol | isotonic agent | 40 mg |
| stevia | sweetening agent | 0.5 mg |
| methylparaben | antiseptic agent | 1 mg |
| purified water | solvent | adjust the whole |
| | | volume to 1 mL |

The viscosity of the suspension measured with a rotatory viscometer (RC-100A, TOKI SANGYO CO., LTD.) was 27 mPa·s. The mean particle size was measured by dispersing the rebamipide suspension in water, with a laser diffraction particle size analyzer (SALD-3000J, Shimadzu Corporation). The mean particle size was 0.17 μm (without ultrasonic irradiation, refractive index: 1.70-0.201).

### Comparative Example 3

20 g of polyvinylpyrrolidone K30 (PVPK30) (BASF) was dissolved in about 400 g of purified water. Thereto, 28.4 g of concentrated hydrochloric acid, and further purified water were added to prepare 550 g of an aqueous solution of PVPK30-hydrochloric acid. Separately, 17.6 g of sodium hydroxide was added to about 2600 g of purified water to prepare an aqueous sodium hydroxide. 81.6 g of rebamipide (Otsuka Pharmaceutical Co., Ltd.) was dissolved in the aqueous sodium hydroxide while warming the solution, and then purified water was added thereto to adjust the total weight to 2940 g. From the prepared sodium hydroxide-rebamipide solution, 1470 g thereof was taken out for the next step.

To the aqueous solution of PVPK30-hydrochloric acid, which was stirred at 3000 rpm with a disperser (ROBOMIX®, PRIMIX Corporation), cooled in ice bath, the above sodium hydroxide-rebamipide solution whose temperature was maintained at about 50° C. was gradually added to precipitate a rebamipide crystal. After all the sodium hydroxide-rebamipide solution was added thereto, the liquid preparation was stirred for 30 minutes. After the liquid preparation was allowed to stand overnight, 5 N aqueous sodium hydroxide was added to the liquid preparation to adjust pH of the liquid preparation to about 5.8.

The resulting aqueous suspension of rebamipide was dispersed for 40 minutes with CLEARMIX W-MOTION (M Technique Co., Ltd.) wherein the rotor was set at about 18000 rpm, and the screen was set at about 16000 rpm. The liquid preparation was concentrated/desalted with a dialyzing system (Pellicon® 2 Mini, MILLIPORE).

After measuring the concentration of rebamipide in the concentrated/desalted sample, polyvinylpyrrolidone K90 (PVPK90) (BASF), stevia (Steviron® C, Morita Kagaku Kogyo Co., Ltd.), D-sorbitol (Wako Pure Chemical Industries, Ltd.), methylparaben, and purified water were added to the sample to prepare 2% rebamipide suspension so that the concentrations of polyvinylpyrrolidone K90, stevia, D-sorbitol, and methylparaben would be 1%, 0.05%, 4%, and 0.1%, respectively.

| Ingredient | | Weight per 1 mL |
|---|---|---|
| rebamipide | active ingredient | 20 mg |
| polyvinyl- | dispersing agent | 10 mg |
| pyrrolidone | | |
| K30 | | |
| polyvinyl- | viscosity enhancing | 10 mg |
| pyrrolidone | agent | |
| K90 | | |
| D-sorbitol | isotonic agent | 40 mg |
| stevia | sweetening agent | 0.5 mg |
| methylparaben | antiseptic agent | 1 mg |
| purified water | solvent | adjust the whole |
| | | volume to 1 mL |

The viscosity of the suspension measured with a rotatory viscometer (RC-100A, TOKI SANGYO CO., LTD.) was 5 mPa·s. The mean particle size was measured by dispersing the rebamipide suspension in water, with a laser diffraction particle size analyzer (SALD-3000J, Shimadzu Corporation). The mean particle size was 0.09 μm (without ultrasonic irradiation, refractive index: 1.70-0.20 i).

### Test 3

As mentioned in Test 1, oral ulcer was induced to the rats, and then the rats were divided in predetermined groups. From the third day after the induction of oral ulcer by cautery method (Day 3), 2% rebamipide suspensions of Examples 3 and 4, 2% rebamipide suspension of Comparative example 3, and each solvent thereof (i.e., each solvent obtained by excluding rebamipide from the Examples and the Comparative example) were intra-orally administered to the rats in a volume of 0.5 mL/kg, four times a day (about at 8:00, 11:00, 14:00 and 17:00) for 5 days. The rats were anesthetized by inhalation of isoflurane and placed in the left lateral decubitus position, and then administered each test sample into the left oral cavity having oral ulcer after making the mouth opened with forceps or a rib retractor.

The area of the oral ulcer was measured on Day 8. The area of the oral ulcer in the group treated with 2% rebamipide suspension of Example 3 was significantly decreased, compared with the group treated with solvent (n=6, p<0.01, t-test). The decrease ratio of the ulcer area in the group treated with the rebamipide suspension was 25.1% per the ulcer area in the group treated with the solvent. In addition, the area of the oral ulcer in the group treated with 2% rebamipide suspension of Example 4 was significantly decreased, compared with the group treated with the solvent (n=6, p<0.01, t-test). The decrease ratio of the ulcer area in the group treated with the rebamipide suspension was 24.8% as compared to the ulcer area in the group treated with the solvent.

On the other hand, the ulcer area in the group treated with the 2% rebamipide suspension of Comparative example 3 was not significantly decreased for the group treated with the solvent (n=6, n.s., t-test). The decrease ratio of the ulcer area in the group treated with the rebamipide suspension was 11.9% as compared to the ulcer area in the group treated with the solvent.

### Example 5

20 g of polyvinylpyrrolidone K30 (PVPK30) (BASF) was dissolved in about 400 g of purified water. Thereto, 28.4 g of concentrated hydrochloric acid, and further purified water were added to prepare 550 g of an aqueous solution of PVPK30-hydrochloric acid. Separately, 8.8 g of sodium hydroxide was added to about 1300 g of purified water to prepare an aqueous sodium hydroxide. 40.8 g of rebamipide (Otsuka Pharmaceutical Co., Ltd.) was dissolved in the aqueous sodium hydroxide while warming the solution, and then purified water was added thereto to adjust the total weight to 1470 g of a solution of sodium hydroxide-rebamipide.

To the aqueous solution of PVPK30-hydrochloric acid, which was stirred at 3000 rpm with a disperser (ROBOMIX®, PRIMIX Corporation), cooled in ice bath, the above sodium hydroxide-rebamipide solution whose temperature was maintained at 50-55° C. was gradually added to precipitate a rebamipide crystal. After all the sodium hydroxide-rebamipide solution was added thereto, the liquid preparation was stirred for 30 minutes. After degassing the liquid preparation, 5 N aqueous sodium hydroxide was added to the liquid preparation to adjust pH of the liquid preparation to about 6.0.

The resulting aqueous suspension of rebamipide was dispersed for 60 minutes with CLEARMIX W-MOTION (M Technique Co., Ltd.) wherein the rotor was set at about 18000 rpm, and the screen was set at about 16000 rpm. The liquid preparation was concentrated/desalted with a dialyzing system (Pellicon® 2 Mini, MILLIPORE).

The concentration of rebamipide in the concentrated/desalted sample was 3.13 w/v %. To 193.6 g of the liquid preparation were added 6 g of polyvinylpyrrolidone K90 (PVPK90) (BASF), 6 g of pullulan (Hayashibara Co., Ltd.), 11.4 g of D-sorbitol (Wako Pure Chemical Industries, Ltd.), 0.21 g of stevia (Steviron® C, Morita Kagaku Kogyo Co., Ltd.), 0.30 g of methyl parahydroxybenzoate (Wako Pure Chemical Industries, Ltd.), and 0.24 g of strawberry flavor (San-Ei Gen F.F.I., Inc.), and then purified water was added thereto to adjust the total volume to 300 mL. After the above additives were completely dissolved, the pH thereof was adjusted to 6.2 with hydrochloric acid or sodium hydroxide.

| Ingredient | | Weight per 1 mL |
|---|---|---|
| rebamipide | active ingredient | 20 mg |
| polyvinyl- | dispersing agent | 10 mg |
| pyrrolidone K30 | | |
| polyvinyl- | viscosity enhancing | 20 mg |
| pyrrolidone K90 | agent | |
| pullulan | viscosity enhancing | 20 mg |
| | agent | |
| D-sorbitol | isotonic agent | 38 mg |
| stevia | sweetening agent | 0.7 mg |
| methyl | preserving agent | 1 mg |
| parahydroxy- | | |
| benzoate | | |
| strawberry | flavor | 0.8 mg |
| flavor | | |
| purified water | solvent | adjust the whole |
| | | volume to 1 mL |

The viscosity of the suspension measured with a rotatory viscometer (RC-100A, TOKI SANGYO CO., LTD.) was 50 mPa·s. The mean particle size was measured by dispersing the rebamipide suspension in water, with a laser diffraction particle size analyzer (SALD-3000J, Shimadzu Corporation). The mean particle size was 0.11 μm (without ultrasonic irradiation, refractive index: 1.70-0.20 i).

### Example 6

10 g of polyvinylpyrrolidone K30 (PVPK30) (BASF) was dissolved in about 400 g of purified water. Thereto, 28.4 g of concentrated hydrochloric acid, and further purified water were added to prepare 550 g of an aqueous solution of PVPK30-hydrochloric acid. Separately, 8.8 g of sodium hydroxide was added to about 1300 g of purified water to prepare an aqueous sodium hydroxide. 40.8 g of rebamipide (Otsuka Pharmaceutical Co., Ltd.) was dissolved in the aqueous sodium hydroxide while warming the solution, and then purified water was added thereto to adjust the total weight to 1470 g of a solution of sodium hydroxide-rebamipide.

To the aqueous solution of PVPK30-hydrochloric acid, which was stirred at 3000 rpm with a disperser (ROBOMIX®, PRIMIX Corporation), cooled in ice bath, the above sodium hydroxide-rebamipide solution whose temperature was maintained at 50-55° C. was gradually added to precipitate a rebamipide crystal. After all the sodium hydroxide-rebamipide solution was added thereto, the liquid preparation was stirred for 30 minutes. After degassing the liquid preparation, 5 N aqueous sodium hydroxide was added to the liquid preparation to adjust pH of the liquid preparation to about 6.0.

The resulting aqueous suspension of rebamipide was dispersed for 60 minutes with CLEARMIX W-MOTION (M Technique Co., Ltd.) wherein the rotor was set at about 18000 rpm, and the screen was set at about 16000 rpm. The liquid preparation was concentrated/desalted with dialyzing system (Pellicon® 2 Mini, MILLIPORE).

The concentration of rebamipide in the concentrated/desalted sample was 4.98 w/v %. To 243.6 g of the liquid preparation were added 6 g of polyvinylpyrrolidone K90 (PVPK90) (BASF), 6 g of pullulan (Hayashibara Co., Ltd.), 11.4 g of D-sorbitol (Wako Pure Chemical Industries, Ltd.), 0.21 g of stevia (Steviron® C, Morita Kagaku Kogyo Co., Ltd.), 0.30 g of methyl parahydroxybenzoate (Wako Pure Chemical Industries, Ltd.), and 0.24 g of strawberry flavor (San-Ei Gen F.F.I., Inc.), and then purified water was added thereto to adjust the total volume to 300 mL. After the above additives were completely dissolved, the pH thereof was adjusted to 6.2 with hydrochloric acid or sodium hydroxide.

| Ingredient | | Weight per 1 mL |
|---|---|---|
| rebamipide | active ingredient | 40 mg |
| polyvinyl- | dispersing agent | 10 mg |
| pyrrolidone K30 | | |
| polyvinyl- | viscosity enhancing | 20 mg |
| pyrrolidone K90 | agent | |
| pullulan | viscosity enhancing | 20 mg |
| | agent | |
| D-sorbitol | isotonic agent | 38 mg |
| stevia | sweetening agent | 0.7 mg |
| methyl | preserving agent | 1 mg |
| parahydroxy- | | |
| benzoate | | |
| strawberry | flavor | 0.8 mg |
| flavor | | |
| purified water | solvent | adjust the whole |
| | | volume to 1 mL |

The viscosity of the suspension measured with a rotatory viscometer (RC-100A, TOKI SANGYO CO., LTD.) was 140 mPa·s. The mean particle size was measured by dispersing the rebamipide suspension in water, with a laser diffraction particle size analyzer (SALD-3000J, Shimadzu Corporation). The mean particle size was 0.17 μm (without ultrasonic irradiation, refractive index: 1.70-0.20 i).

### Example 7

40 g of polyvinylpyrrolidone K30 (PVPK30) (BASF) was dissolved in about 400 g of purified water. Thereto, 28.4 g of concentrated hydrochloric acid, and further purified water were added to prepare 550 g of an aqueous solution of PVPK30-hydrochloric acid. Separately, 8.8 g of sodium hydroxide was added to about 1300 g of purified water to prepare an aqueous sodium hydroxide. 40.8 g of rebamipide (Otsuka Pharmaceutical Co., Ltd.) was dissolved in the aqueous sodium hydroxide while warming the solution, and then purified water was added thereto to adjust the total weight to 1470 g of a solution of sodium hydroxide-rebamipide.

To the aqueous solution of PVPK30-hydrochloric acid, which was stirred at 3000 rpm with a disperser (ROBOMIX®, PRIMIX Corporation), cooled in ice bath, the above sodium hydroxide-rebamipide solution whose temperature was maintained at 50-55° C. was gradually added to precipitate a rebamipide crystal. After all the sodium hydroxide-rebamipide solution was added thereto, the liquid preparation was stirred for 30 minutes. After degassing the liquid preparation, 5 N aqueous sodium hydroxide was added to the liquid preparation to adjust pH of the liquid preparation to about 6.0.

The resulting aqueous suspension of rebamipide was dispersed for 60 minutes with CLEARMIX W-MOTION (M Technique Co., Ltd.) wherein the rotor was set at about 18000 rpm, and the screen was set at about 16000 rpm. The liquid preparation was concentrated/desalted with a dialyzing system (Pellicon® 2 Mini, MILLIPORE).

The concentration of rebamipide in the concentrated/desalted sample was 2.29 w/v %. To 132.1 g of the liquid preparation were added 6 g of polyvinylpyrrolidone K90 (PVPK90) (BASF), 6 g of pullulan (Hayashibara Co., Ltd.), 11.4 g of D-sorbitol (Wako Pure Chemical Industries, Ltd.), 0.21 g of stevia (Steviron® C, Morita Kagaku Kogyo Co., Ltd.), 0.30 g of methyl parahydroxybenzoate (Wako Pure Chemical Industries, Ltd.), and 0.24 g of strawberry flavor (San-Ei Gen F.F.I., Inc.), and then purified water was added thereto to adjust the total volume to 300 mL. After the above additives were completely dissolved, the pH thereof was adjusted to 6.2 with hydrochloric acid or sodium hydroxide.

| Ingredient | | Weight per 1 mL |
|---|---|---|
| rebamipide | active ingredient | 10 mg |
| polyvinyl- | dispersing agent | 10 mg |
| pyrrolidone K30 | | |
| polyvinyl- | viscosity enhancing | 20 mg |
| pyrrolidone K90 | agent | |
| pullulan | viscosity enhancing | 20 mg |
| | agent | |
| D-sorbitol | isotonic agent | 38 mg |
| stevia | sweetening agent | 0.7 mg |
| methyl | preserving agent | 1 mg |
| parahydroxy- | | |
| benzoate | | |
| strawberry | flavor | 0.8 mg |
| flavor | | |
| purified water | solvent | adjust the whole |
| | | volume to 1 mL |

The viscosity of the suspension measured with a rotatory viscometer (RC-100A, TOKI SANGYO CO., LTD.) was 26 mPa·s. The mean particle size was measured by dispersing the rebamipide suspension in water, with a laser diffraction particle size analyzer (SALD-3000J, Shimadzu Corporation). The mean particle size was 0.18 μm (without ultrasonic irradiation, refractive index: 1.70-0.20 i).

### Test 4

As mentioned in Test 1, oral ulcer was induced to the rats, and then the rats were divided in predetermined groups. From the third day after the induction of oral ulcer by cautery method (Day 3), 1%, 2%, and 4% rebamipide suspensions of Examples 7, 5 and 6, respectively, and the solvent thereof (i.e., the solvent obtained by excluding rebamipide from the Example) were intra-orally administered to the rats in a volume of 0.5 mL/kg, four times a day (about at 8:00, 11:00, 14:00 and 17:00) for 5 days. The rats were anesthetized by inhalation of isoflurane and placed in the left lateral decubitus position, and then administered each test sample into the left oral cavity having oral ulcer after making the mouth opened with forceps or a rib retractor.

The area of the oral ulcer was measured on Day 8, and the decrease ratio of the ulcer area in the group treated with the rebamipide suspension as compared to that of the group treated with the solvent was calculated. The area of the oral ulcer in the group treated with 1% rebamipide suspension of Example 7 was decreased, and the area of the oral ulcer in the group treated with 2% and 4% rebamipide suspensions of Examples 5 and 6, respectively was significantly decreased, compared with the group treated with the solvent (n=7, p<0.01, t-test). The decrease ratios of the ulcer area in the group treated with the 1%, 2% and 4% rebamipide suspensions were 13.9%, 25.3% and 33.0% as compared to the ulcer area in the group treated with the solvent, respectively (n=7).

### Example 8

60 g of polyvinylpyrrolidone K30 (PVPK30) (BASF) was dissolved in about 1400 g of purified water. Thereto, 85.2 g of conc. hydrochloric acid solution, and further purified water were added to prepare 1650 g of an aqueous solution of PVPK30-hydrochloric acid. Separately, 26.4 g of sodium hydroxide was added to about 4000 g of purified water to prepare an aqueous sodium hydroxide. 122.4 g of rebamipide (Otsuka Pharmaceutical Co., Ltd.) was dissolved in the aqueous sodium hydroxide while warming the solution, and then purified water was added thereto to adjust the total weight to 4410 g of a solution of sodium hydroxide-rebamipide.

To the aqueous solution of PVPK30-hydrochloric acid, which was stirred with a disperser (CLEARMIX W-MOTION, M Technique Co., Ltd.), cooled in ice bath, wherein the rotor was set at about 6000 rpm, and the screen was set at about 4100 rpm, the above sodium hydroxide-rebamipide solution whose temperature was maintained at 50-55° C. was gradually added to precipitate a rebamipide crystal. After all the sodium hydroxide-rebamipide solution was added thereto, the liquid preparation was stirred for 30 minutes. After degassing the liquid preparation, 5 N aqueous sodium hydroxide was added to the liquid preparation to adjust pH of the liquid preparation to about 6.0.

The resulting aqueous suspension of rebamipide was dispersed for 180 minutes with CLEARMIX W-MOTION (M Technique Co., Ltd.) wherein the rotor was set at about 18100 rpm, and the screen was set at about 16000 rpm. The liquid preparation was concentrated/desalted with a dialyzing system (Pellicon® 2 Mini, MILLIPORE) and filtrated with a filter (Acropak500 capsule 0.8/0.45 μm, PALL).

The concentration of rebamipide in the concentrated/desalted and filtrated sample was 5.10 w/v %. To 792.16 g of the liquid preparation were added 10 g of polyvinylpyrrolidone K90 (PVPK90) (BASF), 20 g of pullulan (Hayashibara Co., Ltd.), 38 g of D-sorbitol (Wako Pure Chemical Industries, Ltd.), 0.7 g of stevia (Steviron® C, Morita Kagaku Kogyo Co., Ltd.), 1.30 g of methyl parahydroxybenzoate (Wako Pure Chemical Industries, Ltd.), 0.55 g of ethyl parahydroxybenzoate (Wako Pure Chemical Industries, Ltd.) and 0.8 g of strawberry flavor (San-Ei Gen F.F.I., Inc.). After the above additives were completely dissolved, the pH thereof was adjusted to 6.2 with sodium hydroxide and then purified water was added thereto to adjust the total volume to 1000 mL.

| Ingredient | | Weight per 1 mL |
|---|---|---|
| rebamipide | active ingredient | 40 mg |
| polyvinyl- | dispersing agent | 20 mg |
| pyrrolidone K30 | | |
| polyvinyl- | viscosity enhancing | 10 mg |
| pyrrolidone K90 | agent | |
| pullulan | viscosity enhancing | 20 mg |
| | agent | |
| D-sorbitol | isotonic agent | 38 mg |
| stevia | sweetening agent | 0.7 mg |
| methyl | preserving agent | 1.3 mg |
| parahydroxy- | | |
| benzoate | | |
| ethyl | preserving agent | 0.55 mg |
| parahydroxy- | | |
| benzoate | | |
| strawberry | flavor | 0.8 mg |
| flavor | | |
| purified water | solvent | adjust the whole |
| | | volume to 1 mL |

The viscosity of the suspension measured with a rotatory viscometer (RC-100A, TOKI SANGYO CO., LTD.) was 37.4 mPa·s. The mean particle size was measured by dispersing the rebamipide suspension in water, with a laser diffraction particle size analyzer (SALD-3000J, Shimadzu Corporation). The mean particle size was 0.23 μm (without ultrasonic irradiation, refractive index: 1.70-0.20 i).

### Test 5

A glossitis was induced by X-ray-irradiation as mentioned below. In detail, the normally-bred rat was anesthetized with intraperitoneal injection of pentobarbital sodium solution. To irradiate only around the snout, the body of the rat was shielded with double shields of 0.5 mm-thick lead plates. The exposed snouts received a single dose of 15 Gy of irradiation. After the X-ray-irradiation, the rat was put back in a breeding cage and was waken therein naturally.

The day of the X-ray-irradiation was defined as the starting day (Day 0).

Eight days before the starting day, the rats were divided in predetermined groups by stratified randomization based on the body weight. From 7 days before the starting day, 1% rebamipide suspension which was prepared by a method equivalent to the method in Example 7 (except for manufacturing scale and the concentrations of methyl parahydroxybenzoate and ethyl parahydroxybenzoate were 0.13% and 0.055%, respectively), 2% rebamipide suspension which was prepared by a method equivalent to the method in Example 5 (except for manufacturing scale and the concentrations of methyl parahydroxybenzoate and ethyl parahydroxybenzoate were 0.13% and 0.055%, respectively), 4% rebamipide suspension of Example 8, and the solvent thereof (i.e., the solvent obtained by excluding rebamipide from the Example) were intra-orally administered to the rats in a volume of 0.5 mL/kg, six times a day for 14 days (up to Day 6).

The X-ray-irradiation was carried out on the starting day (Day 0), and the area of the glossitis on Day 7 was measured. The area of the glossitis in the group treated with rebamipide suspension was decreased dose-dependently, as compared to the area of the glossitis in the group treated with the solvent. The area of the glossitis in the group treated with 1% rebamipide suspension was significantly decreased compared with the group treated with the solvent (n=12, p<0.05, Williams test). And also, the area of the glossitis in the groups treated with 2% and 4% rebamipide suspensions was significantly decreased (n=10-11, p<0.01, Williams test). Each decrease ratio of the glossitis area in the groups treated with the 1%, 2% and 4% rebamipide suspensions was 23.8%, 49.3% and 58.0% as compared to the glossitis area in the group treated with the solvent, respectively.

1. A pharmaceutical composition comprising 10 mg/mL-50 mg/mL rebamipide having a mean particle size of less than 500 nm as an active ingredient, at least one dispersing agent, and at least one viscosity enhancing agent, wherein the viscosity of the liquid preparation is in a range of 10 mPa·s-500 mPa·s.

2. The pharmaceutical composition of claim 1 wherein the mean particle size of rebamipide is less than 300 nm, the content of rebamipide is 20 mg/mL-40 mg/mL, and the viscosity of the liquid preparation is in a range of 20 mPa·s-300 mPa·s.

3. The pharmaceutical composition of claim 1 or 2 wherein the dispersing agent comprises at least one ingredient selected from the group consisting of polyvinylpyrrolidone, hydroxypropylmethylcellulose, polyoxyethylene polyoxypropylene glycol, and carboxymethylcellulose sodium.

4. The pharmaceutical composition of claim 3 wherein the dispersing agent comprises polyvinylpyrrolidone.

5. The pharmaceutical composition of claim 4 wherein the dispersing agent comprises polyvinylpyrrolidone K25 and/or polyvinylpyrrolidone K30.

6. The pharmaceutical composition of any one of claims 1-5 wherein the viscosity enhancing agent comprises polyvinylpyrrolidone K90.

7. The pharmaceutical composition of any one of claims 1-5 wherein the viscosity enhancing agent comprises pullulan.

8. The pharmaceutical composition of any one of claims 1-5 wherein the viscosity enhancing agent comprises polyvinylpyrrolidone K90 and pullulan.

9. The pharmaceutical composition of claim 8 wherein the viscosity enhancing agent comprises 5 mg/mL-30 mg/mL polyvinylpyrrolidone K90 and 10 mg/mL-30 mg/mL pullulan.

10. The pharmaceutical composition of any one of claims 1-9 wherein the viscosity enhancing agent has no aggregative action for the rebamipide particles.

11. The pharmaceutical composition of any one of claims 1-9 which is prepared via the following steps
mixing at least one dispersing agent, an aqueous acid solution, an aqueous solution comprising a water-soluble rebamipide salt, and optional other ingredient(s), or solvent(s) to prepare an aqueous suspension comprising rebamipide having a mean particle size of less than 500 nm, and then
adding a viscosity enhancing agent thereto.

12. The pharmaceutical composition of claim 11 which is prepared via the following steps
mixing at least one dispersing agent, an aqueous acid solution, an aqueous solution comprising a water-soluble rebamipide salt, and optional other ingredient(s) or solvent(s) to prepare an aqueous suspension comprising rebamipide having a mean particle size of less than 500 nm,
adding a base thereto to adjust pH of the aqueous suspension to 3-7,
dispersing and/or dialyzing the aqueous suspension,
adjusting pH of the aqueous suspension to 5-7, and then
adding a viscosity enhancing agent thereto.

13. The pharmaceutical composition of any one of claims 1-12 wherein the mean particle size of rebamipide is less than 200 nm.

14. The pharmaceutical composition of any one of claims 1-13 wherein the shape of rebamipide is a uniform needle crystal having the longest diameter of less than 1000 nm and the shortest diameter of less than 60 nm, provided that the longest diameter/the shortest diameter is more than 3.

15. The pharmaceutical composition of any one of claims 1-14, further comprising a parahydroxybenzoate derivative as a preserving agent (antiseptic agent).

16. The pharmaceutical composition of any one of claims 1-15, further comprising an isotonic agent, a sweetening agent, and/or a flavor.

17. The pharmaceutical composition of claim 16 comprising stevia as a sweetening agent.

18. The pharmaceutical composition of any one of claims 1-17 which is in the form of aqueous suspension.

19. A method for preparing the pharmaceutical composition of any one of claims 1-18, comprising
mixing at least one dispersing agent, an aqueous acid solution, an aqueous solution comprising a water-soluble rebamipide salt, and optional other ingredient(s) or solvent(s) to prepare an aqueous suspension comprising rebamipide having a mean particle size of less than 500 nm,
adding a base thereto to adjust pH of the aqueous suspension to 3-7,
dispersing and/or dialyzing the aqueous suspension,
adjusting pH of the aqueous suspension to 5-7, and then
adding a viscosity enhancing agent thereto and optionally adding a preserving agent (antiseptic agent), an isotonic agent, a sweetening agent, and/or a flavor thereto.

20. A method for preventing and/or treating mucousal disorder in the oral cavity, comprising administering the pharmaceutical composition of any one of claims 1-18 in the oral cavity.

21. A method for preventing and/or treating mucosal disorder in the oral cavity and/or mucosal disorder in the pharynx, comprising administering 3 mL-20 mL of the pharmaceutical composition of any one of claims 1-18 in the oral cavity and then getting the patient to swallow the pharmaceutical composition.

22. The method of claim 21 wherein the mucosal disorder in the oral cavity and/or mucosal disorder in the pharynx is caused by radiation and chemotherapy, and the amount of the pharmaceutical composition administered in the oral cavity is 5 mL-10 mL.

23. A method for preventing and/or treating mucosal disorder in the oral cavity, comprising repeating the operation of the method defined in claim 21 or 22 twice to six times a day.

24. A method for preventing and/or treating mucosal disorder in the oral cavity and/or mucosal disorder in the pharynx caused by radiation and chemotherapy, comprising repeating the operation of the method defined in claim 21 or 22 twice to six times a day.

25. A method for preventing and/or treating xerostomia and/or hyposalivation, comprising administering the pharmaceutical composition of any one of claims 1-18 in the oral cavity.
